(19) United States
(12) Patent Application Publication
(10) Pub. No.: US 2025/0146939 A1
(43) Pub. Date: May 8, 2025

(54) SERS NANO-PARTICLE, PREPARATION METHOD THEREFOR, AND APPLICATION THEREOF TO METHOD FOR DISTINGUISHING CTCS FROM WBCS

(71) Applicants: NINGBO CIXI INSTITUTE OF BIOMEDICAL ENGINEERING, Ningbo (CN); NINGBO INSTITUTE OF MATERIALS TECHNOLOGY AND ENGINEERING, CHINESE ACADEMY OF SCIENCES, Ningbo (CN); ZHEJIANG CANCER HOSPITAL, Hangzhou (CN)

(72) Inventors: Aiguo WU, Ningbo (CN); Jie LIN, Ningbo (CN); Dinghu Zhang, Ningbo (CN); Xiaoxia WU, Ningbo (CN); Guoliang SHAO, Ningbo (CN)

(21) Appl. No.: 18/838,080

(22) PCT Filed: Feb. 15, 2023
(86) PCT No.: PCT/CN2023/076038

§ 371 Date: Aug. 13, 2024

(30) Foreign Application Priority Data
Feb. 16, 2022 (CN) 202210148829.0
Apr. 21, 2022 (CN) 202210425260.8
Jun. 1, 2022 (CN) 202210626736.4

(51) Int. Cl. G01N 21/64; C07K 16/30; G01N 33/569; G01N 33/574

(52) U.S. Cl. CPC G01N 21/6486; C07K 16/30; G01N 33/56972; G01N 33/574; G01N 2201/129; G01N 2333/705

(57) ABSTRACT

Disclosed are a SERS nano-particle, a preparation method therefor, and an application thereof to a method for distinguishing CTCs from WBCs. The SERS nano-particle are formed by core magnetic particles, noble metal nano-particles, Raman signal molecules, hydrophilic molecules and target molecules. A method for distinguishing CTCs from WBCs comprises: enabling a SERS nano-particle to make contact with a to-be-detected solution containing CTCs and/or WBCs; performing incubation; then detecting SERS signal intensity of a cell combined with the SERS nano-particle; setting SERS signal intensity threshold; if SERS signal intensity of the cell combined with the SERS nano-particle exceeds SERS signal intensity threshold, determining the cell as CTS; otherwise, determining the cell as WBC. A ROC curve is used for the first time to assist the SERS technique in distinguishing CTCs from WBCs.

### TECHNICAL FIELD

The invention belongs to the technical field of materials, and relates to a SERS nano-particle, a preparation method therefor, and an application thereof to a method for distinguishing CTCs from WBCs.

### DESCRIPTION OF RELATED ART

Circulating tumor cells (CTCs) represent a transition state of tumor metastasis and carry abundant biological information related to primary tumors and metastatic tumors. The CTCs, as a noninvasive liquid biopsy method, are of great clinical significance for early in-vitro diagnosis, evaluation of the drug resistance, and determination of prognosis and survival time of patients. However, the number of CTCs in blood is extremely small, only several to dozens of CTCs in each milliliter of blood. CTC capture techniques include density gradient precipitation, size-exclusion filtration, self-driven micro-machines, magnetic beads, micro-fluidic chips, and the like. At present, the unique technique that has been approved by FDA is EpCAM-based immunomagnetic bead capture such as a CellSearch capture system. However, in the tumor metastasis process, tumors often undergo epithelial-mesenchymal transition (EMT), which will lead to a loss of EpCAM molecules, thus increasing the missed detection rate of tumor cells.

Trop2, as a transmembrane glycoprotein, has an expression in tumors of many cancers such as liver cancer, lung cancer, breast cancer and stomach cancer, and particularly, the trop2 expression of over 90% of patients suffering from triple negative breast cancer (TNBC) is positive. At present, there has been no study on the application of trop2 antibodies to CTCs.

In addition to capture, the identification of captured CTCs and white blood cells (WBCs) entrained during CTC capture, is another major challenge of CTC detection: a surface-enhanced Raman scattering (SERS) probe is bound to a corresponding receptor on the surface of a tumor cell by means of the specificity of an antibody coupled thereto, and then tumor cells are recognized by detecting Raman signals of the SERS probe targeted to the surface of cells. However, because WBCs in blood also carry Raman signals due to nonspecific adsorption of the SERS probe, the recognition of CTCs will be seriously affected. Although some SERS probes made from low-adsorption materials reduce the binding to normal blood cells, they cannot completely eliminate such a combination. Therefore, under the precondition that the nonspecific adsorption is inevitable, how to better distinguish tumor cells from normal blood cells is an important issue urgently to be settled in the field of SERS-based CTC detection.

Receiver operating characteristic (ROC) curve analysis is a method widely used for evaluating the performance of diagnostic tests. The ROC curve not only can be used for evaluating the overall diagnostic capacity of a test, but also can be used for determining corresponding diagnostic critical values of the sensitivity and specificity. However, there has not been any report about the use of the ROC curve for SERS-based CTC detection.

### BRIEF SUMMARY OF THE INVENTION

In view of the defects in the prior art, the invention aims to provide a SERS nano-particle, a preparation method therefor, and an application of the SERS nano-particle and a ROC curve to a method for distinguishing CTCs from WBCs.

One objective of the invention is to provide a SERS nano-particle formed by core magnetic particles, noble metal nano-particles, Raman signal molecules, hydrophilic molecules and target molecules.

In the SERS nano-particle, the magnetic particles comprise at least one selected from Fe nano-particles, FeO nano-particles and Fe₃O₄ nano-particles, and are preferably the Fe₃O₄ nano-particles.

In the SERS nano-particle, the noble metal nano-particles comprise at least one selected from gold particles, silver particles, platinum particles and copper particles, preferably comprise at least one selected from gold nano-particles, silver nano-particles, platinum nano-particles and copper nano-particles, and are preferably the gold particles, further preferably the gold nano-particles.

In the SERS nano-particle, the Raman signal molecules comprise at least one selected from 4-mercaptobenzoic acid (4-MBA), mercaptopyridine, 4-aminothiophenol, naphthalenethiol, 4-fluorothiophenol, rhodamine, crystal violet, alizarin red and Nile blue, and are preferably the 4-MBA.

In the SERS nano-particle, the hydrophilic molecules comprise at least one selected from polydopamine (PDA), bovine serum albumin and polyethylene glycol, and are preferably the PDA.

In the SERS nano-particle, the target molecules comprise at least one selected from antibodies anti-trop2, anti-EGFR, anti-EpCAM and anti-Her2, and are preferably the antibody anti-trop2. Trop2 has a high expression in TNBC cells, has no expression in normal blood cells, and will not be lost with EMT, so it is a promising CTC capture strategy for capturing most solid tumors, especially NTBC CTCs, by means of nano-particles modified by the antibody trop2.

Optionally, the mass percentage of the target molecules in the SERS nano-particle is 0.01/6-1%.

Optionally, the SERS nano-particle sequentially comprises, from inside to outside, core particles, a noble metal nano-particle layer, a Raman signal molecular layer, a high molecular layer and a target antibody anti-trop2 layer;
the core particles are magnetic particles, surfaces of which are provided with a positive-charge polymer modifying layer;
the noble metal nano-particle layer is layered structure formed by noble metal nano-particles assembled on the surfaces of the core particles by means of electrostatic interaction;
the Raman signal molecular layer is a layered structure formed by Raman signal molecules connected to a surface of the noble metal nano-particle layer;
the high molecular layer is a layered structure formed by hydrophilic molecules wrapping a surface of the Raman signal molecular layer;
the target antibody anti-trop2 layer is a layered structure formed by a target antibody anti-trop2 coupled to an outer surface of the high molecular layer.

Optionally, a positive-charge polymer in the positive-charge polymer modifying layer comprises polyetherimide (PEI).

In the SERS nano-particle, the particle size of the core particles is 10-1000 nm. Optionally, the particle size of the core particles is any one of or within a range between any two of 10 nm, 20 nm, 50 nm, 150 nm, 200 nm, 250 nm, 300 nm, 500 nm, 800 nm and 1000 nm.

In the SERS nano-particle, the particle size of the noble metal nano-particles is 1-100 nm. Optionally, the particle size of the noble metal nano-particles is any one of or within a range between any two of 1 nm, 20 nm, 40 nm, 60 nm, 80 nm and 100 nm.

The particle size of the SERS nano-particle is 100-1000 nm. Optionally, the particle size of the SERS nano-particle is any one of or within a range between any two of 10 nm, 20 nm, 50 nm, 150 nm, 200 nm, 250 nm, 300 nm, 500 nm, 800 nm and 1000 nm.

In the SERS nano-particle, the mass ratio of the magnetic particles, the positive-charge polymer modifying layer, the noble metal nano-particle layer, the Raman signal molecular layer, the high molecular layer and the target antibody anti-trop2 layer is 70-120:5-40:10-40:2-10:1-20:0.01-10.

Another objective of the invention is to provide a preparation method for the SERS nano-particle, comprising the following steps:
(S1) obtaining core particles;
(S2) assembling noble metal nano-particles on surfaces of the core particles by means of electrostatic interaction to form a noble metal nano-particle layer to obtain a particle I;
(S3) connecting Raman signal molecules to a surface of the noble metal nano-particle layer to form a Raman signal molecular layer to obtain a particle II;
(S4) wrapping a surface of the Raman signal molecular layer with hydrophilic molecules to form a high molecular layer to obtain a particle III; and
(S5) coupling an antibody anti-trop2 to a surface of the high molecular layer to form a target antibody anti-trop2 layer to obtain a magnetic SERS nano-particle.

In the preparation method for the SERS nano-particle, in (S1), the core particles are obtained by a reaction I of a solution containing a magnetic metal salt, an alkaline substance, a positive-charge polymer and a solvent I.

Optionally, the magnetic metal salt comprises a magnetic metal chloride, and preferably comprises FeCl₃·6H₂O.

Optionally, the alkaline substance comprises CH₃COONa.

Optionally, the positive-charge polymer comprises PEI.

Optionally, the solvent I comprises glycol.

Optionally, the ratio of the magnetic metal salt, the alkaline substance, the positive-charge polymer and the solvent I is 0.02-5 g:0.5-10 g:0.01-8 g:1-80 ml. Preferably, the ratio of the magnetic metal salt, the alkaline substance, the positive-charge polymer and the solvent I is 0.02-2 g:0.5-10 g:0.01-5 g:1-50 ml. Preferably, the ratio of the magnetic metal salt, the alkaline substance, the positive-charge polymer and the solvent I is 0.02-1 g:0.5-5 g:0.01-2 g:1-30 ml.

Optionally, conditions for the reaction I are as follows:
the time for the reaction I is 1-10 hrs;
the temperature for the reaction I is 100-500° C.

Optionally, the time for the reaction I is any one of or within a range between any two of 1 h, 2 hrs, 3 hrs, 5 hrs, 8 hrs and 10 hrs, and the temperature for the reaction I is any one of or within a range between any two of 100° C., 180° C., 220° C., 250° C., 300° C. and 500° C.

In the preparation method for the SERS nano-particle, in (S2), a noble metal nano-particle solution and a core particle solution are mixed, and then stirring I is performed to obtain the particle I.

Optionally, the concentration of the noble metal nano-particle solution is 0.1-3 mg/ml, and the concentration of the core particle solution is 1-5 mg/ml.

Optionally, the concentration of the noble metal nano-particle solution is any one of or within a range between any two of 0.1 mg/ml, 0.6 mg/ml, 1 mg/ml, 1.2 mg/ml, 1.5 mg/ml, 2 mg/ml and 3 mg/ml; and the concentration of the core particle solution is any one of or within a range between any two of 1 mg/ml, 2 mg/ml, 2.5 mg/ml, 3 mg/ml, 4 mg/ml and 5 mg/ml.

Optionally, the volume ratio of the noble metal nano-particle solution and the core particle solution is 1-50:0.5-20; preferably, the volume ratio of the noble metal nano-particle solution and the core particle solution is 1-30:0.5-10; preferably, the volume ratio of the noble metal nano-particle solution and the core particle solution is 1-10:0.5-20; and preferably, the volume ratio of the noble metal nano-particle solution and the core particle solution is 1-10:0.5-5.

Optionally, the time for the stirring I is 10-120 min; and optionally, the time for the stirring I is any one of or within a range between any two of 10 min, 20 min, 30 min, 40 min, 50 min, 80 min and 120 min.

Optionally, the noble metal nano-particles are obtained by the following steps:
adding a noble metal salt solution to water, heating to boiling, adding a reducer solution, and further heating for 3-360 min.

The noble metal salt solution is added to water and heated to boiling, the reducer solution is added, and a mixed solution is further heated for a time, which is any one of or within a range between any two of 3 min, 10 min, 20 min, 30 min, 50 min, 100 min, 150 min, 200 min, 250 min and 360 min.

Optionally, a noble metal salt comprises HAuCl₄·4H₂O, and a reducer comprises sodium citrate.

Optionally, the concentration of the noble metal salt solution is 1-100 mM; and optionally, the concentration of the noble metal salt solution is any one of or within a range between any two of 1 mM, 5 mM, 10 mM, 15 mM, 20 mM, 50 mM, 80 mM and 100 mM.

Optionally, the concentration of the reducer solution is 0.1-10 wt %; and optionally, the concentration of the reducer solution is any one of or within a range between any two of 0.1 wt %, 1 wt %, 5 wt %, 8 wt % and 10 wt %.

Optionally, the volume ratio of the noble metal salt solution, the water and the reducer solution is 1-20:1-100:0.05-5.

Optionally, the heating is performed at a temperature of 100-200° C.

In the preparation method for the SERS nano-particle, in (S3), a Raman signal molecule solution and a particle I solution are mixed, and then stirring II is performed to obtain the particle II.

Optionally, the concentration of the Raman signal molecule solution is 1-100 mM; and optionally, the concentration of the Raman signal molecule solution is any one of or within a range between any two of 1 mM, 5 mM, 10 mM, 30 mM, 50 mM, 80 mM and 100 mM. Optionally, the concentration of the particle I solution is 0.1-8 mg/mL.

Optionally, the volume ratio of the Raman signal molecule solution and the particle I solution is 160-480 μl:16-500 ml; preferably, the volume ratio of the Raman signal molecule solution and the particle I solution is 160-480 μl:16-100 ml; and preferably, the volume ratio of the Raman signal molecule solution and the particle I solution is 160 μl:16-500 ml.

Optionally, the time for the stirring II is 1-10 hrs; and optionally, the time for the stirring II is any one of or within a range between any two of 1 h, 3 hrs, 5 hrs, 8 hrs and 10 hrs.

In the preparation method for the SERS nano-particle, in (S4), a particle II solution and CH₃CH₂OH, NH₃·H₂O are mixed, stirring III is performed, and a polydopamine solution is added for a reaction III to obtain the particle III.

Optionally, the concentration of the particle II solution is 0.01-10 mg/ml; and optionally, the concentration of the particle II solution is any one of or within a range between any two of 0.01 mg/ml, 0.19 mg/ml, 0.3 mg/ml, 0.5 mg/ml, 1 mg/ml, 3 mg/ml, 5 mg/ml, 8 mg/ml and 10 mg/ml.

Optionally, the concentration of the polydopamine solution is 1-100 mg/ml; and optionally, the concentration of the polydopamine solution is any one of or within a range between any two of 1 mg/ml, 20 mg/ml, 30 mg/ml, 40 mg/ml, 50 mg/ml, 60 mg/ml and 100 mg/ml.

The effect of the SERS nano-particle can be better improved by controlling the concentration of the polydopamine solution.

Optionally, the ratio of the particle II solution, the CH₃CH₂OH, NH—H₂O and the polydopamine solution is 6-54 ml:2-16 ml:300-1200 μl:0.4-10 ml.

Optionally, the time for the stirring III is 1-100 min; and optionally, the time for the stirring III is any one of or within a range between any two of 1 min, 10 min, 20 min, 30 min, 50 min, 80 min and 100 min.

Optionally, the time for the reaction III is 1-10 hrs; and optionally, the time for the reaction III is any one of or within a range between any two of 1 h, 3 hrs, 5 hrs, 8 hrs and 10 hrs.

In the preparation method for the SERS nano-particle, in (S5), the SERS nano-particle is obtained by stirring IV of a solution containing the particle III, a buffer solution and a target antibody anti-trop2.

Optionally, the buffer solution comprises at least one selected from a Tris-HCl solution and an ammonia alkaline solution.

Optionally, the ratio of the particle III, the buffer solution and the target antibody anti-trop2 is 0.05-10 mg:1-100 ml:0.05-50 μg.

Optionally, the time for the stirring IV is 1 h-72 hrs; and optionally, the time for the stirring IV is any one of or within a range between any two of 1 h, 2 hrs, 10 hrs, 12 hrs, 15 hrs, 20 hrs, 30 hrs, 50 hrs and 72 hrs.

Optionally, the temperature for the stirring IV is 15-40° C.; and optionally, the temperature for the stirring IV is any one of or within a range between any two of 15° C., 20° C., 25° C., 30° C., 35° C. and 40° C.

Another objective of the invention is to provide a method for distinguishing CTCs from WBCs for a non-diagnostic purpose, comprising: enabling a SERS nano-particle to make contact with a to-be-detected solution containing CTCs and/or WBCs; performing incubation; then detecting a SERS signal intensity of a cell combined with the SERS nano-particle; setting a SERS signal intensity threshold; if the SERS signal intensity of the cell combined with the SERS nano-particle exceeds the SERS signal intensity threshold, determining the cell as a CTS; otherwise, determining the cell as a WBC.

The method specifically comprises: enabling a SERS nano-particle to make contact with a to-be-detected solution containing CTCs and/or WBCs; performing incubation; then detecting a SERS signal intensity of a cell combined with the SERS nano-particle to obtain the SERS signal intensity In of each cell; comparing the SERS signal intensity In of each cell with the SERS signal intensity threshold I;
if In>I, determining the corresponding cell as a CTC; or
if In≤I, determining the corresponding cell as a WBC.

In the method, the SERS signal intensity threshold is a point value within a fluorescence intensity of 0-3000a.u.

In the method, the SERS signal intensity threshold is determined according to a ROC curve of SERS intensities of CTCs and WBCs in known samples.

In the method, SERS intensities of CTCs and WBCs in known samples are input to a SPSS, and the SERS signal intensity threshold is obtained after analysis of a ROC curve.

The SERS intensities of CTCs and WBCs in known samples are input to the SPSS to plot a ROC curve, a series of cut-off values corresponding to different sensitivities and specificities are obtained after the ROC curve is analyzed, and a cut-off value corresponding to a maximum specificity and sensitivity is the SERS signal intensity threshold.

According to the method provided by the invention, SERS and the ROC curve are used together for the first time to detect CTCs in peripheral blood samples, the SERS signal intensity is used in conjunction with the threshold of the ROC curve, and tumor cells and WBCs are distinguished by means of the ROC curve for comparing the SERS intensity of CTCs and the SERS intensity of the WBCs, such that Raman signal interference of WBCs in CTC detection can be eliminated to the maximum extent. The unique recognition method based on SERS signal-ROC curve can realize accurate diagnosis of CTCs, and this new strategy has a great potential in detection of CTCs in true blood.

Another objective of the invention is to provide a system for distinguishing CTCs from WBCs for a non-diagnostic purpose, comprising:
a sample loading module, used for collecting and/or storing samples, wherein the samples comprise known samples formed by certain CTCs and WBCs and/or to-be-detected samples formed by uncertain CTCs and WBCs;
an ROC curve construction module, used for detecting SERS signal intensities of the known samples, plotting an ROC curve of specificity and sensitivity, and outputting a recommended threshold (cut-off value) according to detection requirements; and
a detection module, used for detecting SERS signal intensities of the to-be-detected samples and outputting a detection result identifier according to the set threshold.

If the SERS signal intensity of the to-be-detected samples is greater than the cut-off value, the detection module outputs a CTC result identifier; or, if the SERS signal intensity of the to-be-detected sample is less than or equal to the cut-off value, the detection module outputs a WBC result identifier.

Compared with the prior art, the invention has the following beneficial effects:
1. The SERS nano-particle provided by the invention comprises core magnetic particles, noble metal nano-particles, Raman signal molecules, hydrophilic molecules and target molecules, thus being able to realize efficient capture and specific recognition of CTCs; the use of the magnetic particles and the noble metal nano-particles enhances SERS signals;
2. The SERS nano-particle provided by the invention preferably has a target antibody anti-trop2 layer on the surface and uses trop2 as a target antibody for capturing CTCs for the first time, thus being able to realize efficient capture and sensitivity detection of CTCs with a positive trop2 expression;
3. In the invention, the ROC curve is used for the first time to assist the SERS technique in distinguishing CTCs from WBCs, and the capacity to recognize tumor cells and the threshold of the SERS intensity can be determined by means of the ROC curve for comparing the SERS intensity of tumor cells with the SERS intensity of WBCs;
4. According to the method for distinguishing CTCs from WBCs provided by the invention, different detection purposes, for example, accurate diagnosis of tumor cells for gene detection of the tumor cells or primary screening of tumor cells for early warning of tumor recurrence, can be fulfilled by selecting the SERS signal intensity threshold;
5. According to the invention, by setting the SERS signal intensity threshold, Raman signal interference of WBCs in CTC detection can be eliminated to the maximum extent to realize accurate diagnosis of CTCs, and the invention has a great potential in detecting CTCs in true blood.

### BRIEF DESCRIPTION OF THE SEVERAL VIEWS OF THE DRAWINGS

FIG. 1A illustrate an SEM image of Fe₃O₄ NPs prepared in Embodiment 1, FIG. 1B is an SEM image of Au NPs prepared in Embodiment 1, FIG. 1C is an SEM image of Fe₃O₄@Au NPs prepared in Embodiment 1, and FIG. 1D is an SEM image of Fe₃O₄@Au NPs-MBA@PDANPs prepared in Embodiment 1;

FIG. 2 illustrates confocal images obtained after reaction of Fe3O4@Au NPs-MBA@PDA-anti-trop2 NPs and donkey anti-rabbit IgG with green fluorescence according to Embodiment 2, wherein FIG. 2A is a bright-field image, FIG. 2B is a fluorescence image, and FIG. 2C is an image obtained by combining FIG. 2A and FIG. 2B;

FIG. 3 illustrates the cancer cell capture efficiency of Fe₃O₄@Au NPs-MBA@PDA-anti-trop2 NPs according to Embodiment 3;

FIG. 4 illustrates SERS intensities obtained after co-incubation of three types of tumor cell lines and Fe₃O₄@Au NPs-MBA@PDA-anti-trop2 NPs according to Embodiment 4, wherein A illustrates a corresponding SERS spectrogram of the three types of tumor cell lines and WBCs, and B illustrates the SERS intensity of each of four types of cells;

FIG. 5 illustrates a ROC curve for comparing SERS intensities of MDA-MB-231, MDA-MB-468, HCC1806 and WBCs according to Embodiment 5.

### DETAILED DESCRIPTION OF THE INVENTION

The technical solutions of the invention are further described below with reference to specific embodiments and accompanying drawings. It should be understood that the specific embodiments described here are merely used for helping understand the invention and are not intended to specifically limit the invention. In addition, the drawings used here are merely for better explaining the contents disclosed by the invention and should not be construed as limitations of the protection scope of the invention. Unless otherwise specially stated, raw materials used in the embodiments of the invention are all common raw materials in the art, and methods adopted in the embodiments are conventional methods in the art.

In one specific embodiment of the invention, a SERS nano-particle for detecting CTCs in most solid tumors, particularly CTCs in TNBC tumors, is provided. The SERS nano-particle comprises: magnetic nano-particles Fe₃O₄ NPs used as a core; noble metal nano-particles Au NPs, the noble metal nano-particles Au NPs being assembled on surfaces of the magnetic nano-particles Fe₃O₄ NPs, and Raman signal molecules 4-MBA being fixed to surfaces of the noble metal nano-particles Au NPs; hydrophilic molecules PDA, the hydrophilic molecules PDA having multiple functional groups bound to the noble metal nano-particles Au NPs and being spontaneously polymerized in an alkaline environment to wrap the surfaces of the noble metal nano-particles Fe₃O₄@AuNPs-MBA NPs; and a target antibody anti-trop2, the target antibody anti-trop2 being coupled to the hydrophilic molecules PDA.

In another specific embodiment of the invention, a preparation method for the SERS nano-particle is provided. The preparation method for the SERS nano-particle comprises: first, preparing, by a hydrothermal method, magnetic Fe₃O₄ nano-particles, surfaces of which are modified with PEI (surfaces of the magnetic Fe₃O₄ nano-particles are provided with positive charges); second, preparing, by a reduction method, gold nano-particles (surfaces of which are provided with negative charges) with sodium citrate; third, self-assembling noble metal nano-particles AuNPs on surfaces of the Fe₃O₄ nano-particles by means of electrostatic interaction, and self-assembling gold nano-particles AuNPs to realize electromagnetic field superposition between adjacent gold nano-particles to form hot spots to enhance SERS signals; then, modifying surfaces of composite nano-particles with Raman signal molecules 4-MBA to allow composite beads to have SERS signals; next, wrapping Fe₃O₄@AuNPs-MBA with a PDA shell to improve the stability of the Fe₃O₄@AuNPs-MBA and provide a stent to which a target antibody anti-trop2 adheres; and finally, coupling the target antibody anti-trop2 to surfaces of Fe₃O₄@Au NPs-MBA@PDA NPs. The SERS nano-particle is used for CTC detection and can realize efficient capture and specific recognition of tumor cells with a positive trop2 expression.

In another specific embodiment of the invention, a method for distinguishing CTCs from WHCs by means of an ROC curve and a SERS technique is provided. First, a SERS nano-particle is prepared, and with TNBC cells with different trop2 expressions as model cells, the SERS nano-particle can effectively capture tumor cells with a positive trop2 expression and endow the tumor cells with Raman signals. For the recognition of tumor cells, a SERS intensity threshold for recognizing tumor cells is determined by means of a ROC curve for comparing the SERS intensities of TNBC cells (HCC1806, MDA-MB-468, MDA-MB-231) and WBCs. The most important characteristic of the threshold is that it can be selected according to different proposes of tumor cell recognition. In a case where tumor cells need to be accurately diagnosed for gene detection of the tumor cells, the threshold (cut-off value) may be set to 281, the specificity is 100% (the misdiagnosis rate is 0), and the sensitivity is 69%; or, in a case where tumor cells need to be primarily screened for early warning of tumor recurrence, the threshold (cut-off value) may be set to 206, the specificity is 90%, and the sensitivity is 76%. By means of such a unique recognition method, tumor cells can be primarily screened and/or accurately diagnosed, and the SERS nano-particle with high capture efficiency has a great potential in detecting CTCs in true blood. The method for recognizing CTCs provided by the invention does not need to deliberately avoid SERS signals of WBCs and provides a new angle of view for CTC recognition based on the SERS technique.

Some embodiments of the invention are described in detail below in conjunction with accompanying drawings. The following embodiments and features in the embodiments can be combined without conflicts.

### Embodiment 1

(1) Preparation of Fe₃O₄ NPs

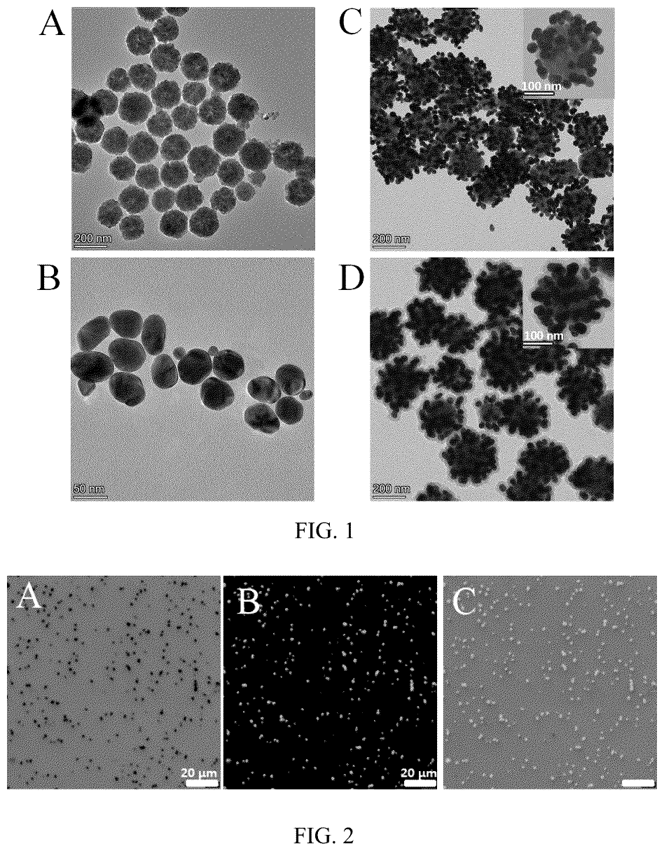

0.68 g of FeCl₃·6H₂O, 1.8 g of CH₃COONa and 0.75 g of PEI were sequentially added to 20 ml of glycol and then continuously stirred at a temperature of 60° C. until all substances were completely dissolved. A mixed solution was transferred into a reactor, heated to 220° C. for reaction for 2 hrs, cooled to room temperature, then washed three times respectively with ethyl alcohol and deionized water, and finally dispersed in 50 mL of deionized water, and then centrifuged for 5 min to obtain supernatant. It can be seen, from FIG. 1A which illustrates an SEM image of prepared Fe₃O₄ NPs, that the surface of the Fe₃O₄NPs is modified with PEI and the particle size of Fe₃O₄ NPs-PEI is about 150 nm.

### (2) Preparation of AuNPs

10 ml of 5 mM HAuCl₄·4H₂O was added to 40 ml of deionized water and heated by a 150° C. oil bath to be boiled, then 2.3 ml of a 1% citric acid solution (w/w) was added quickly for further reaction for 20 min, then heating was stopped, and the mixed solution was cooled to room temperature. As shown in FIG. 1B, which illustrates an SEM image of prepared AuNPs, the particle size of the AuNPs is about 40 nm.

(3) Preparation of Fe₃O₄@Au NPs

12 ml of a AuNPs solution (solvent: water; concentration: 1.2 mg/ml) and 1.6 ml of a Fe₃O₄ NPs solution (solvent: water; concentration: 2.3 mg/ml) were mixed, then stirred for 30 min with a teflon rod, washed with deionized water, and dispersed in 16 ml of deionized water to obtain Fe₃O₄@Au NPs. As shown in FIG. 1C, which illustrates an SEM image of prepared Fe₃O₄@Au NPs, the particle size of the Fe₃O₄@Au NPs of a core-shell structure is about 200 nm.

(4) Preparation of Fe₃O₄@Au-MBA NPs

160 ul of 1 mM 4-MBA ethanol solution was added to 16 ml of a Fe₃O₄@Au solution (solvent: water; concentration: 0.21 mg/ml), a mixed solution was stirred with a teflon rod for 2 hrs, sufficiently washed with deionized water and finally dispersed in 18 ml of a deionized water solution, and a SERS signal of the mixed solution was measured.

(5) Preparation of Fe₃O₄@Au NPs-MBA@PDA NPs

18 ml of a Fe₃O₄@Au-MBA solution (solvent: water; concentration: 0.19 mg/ml), 8 ml of CH₃CH₂OH and 600 μl of NH₃·H₂O were mixed and then stirred with a teflon rod for 20 min, then 2 ml of a polydopamine solution (40 mg/ml) was slowly added to a mixed solution, and the mixed solution was sufficiently washed with deionized water 5 hrs later and dissolved in 8 ml of deionized water. As shown in FIG. 1D, which illustrates an SEM image of prepared Fe₃O₄@Au NPs-MBA@PDA NPs, that a 10 nm polydopamine layer wraps the surface of AU, and the particle size of the Fe₃O₄@Au NPs-MBA@PDA NPs is about 200 nm.

(6) Preparation of Fe₃O₄@Au NPs-MBA@PDA-Anti-Trop2 NPs

4 ml of Fe₃O₄@Au NPs-MBA@PDA was obtained and attracted with a magnet, supernatant was removed, then the Fe₃O₄@Au NPs-MBA@PDA was added to 4 ml of a Tris-HCl solution (10 mM, PH=8.5), then 40 μg of an antibody anti-trop2 was added, and a mixed solution was stirred at room temperature for 12 hrs, washed with PBS three times, and finally disposed in 4 ml of a PBS solution. The particle size of Fe₃O₄@Au NPs-MBA@PDA-anti-trop2 NPs is about 200 nm.

### Embodiment 2

To verify the target antibody anti-trop2 was connected to the surface of Fe₃O₄@Au NPs-MBA@PDA NPs, 2 μg of Alex488-labeled donkey anti-rabbit IgG (ThermoFisher, MA5-29829) and 1.4 mg of Fe₃O₄@Au NPs-MBA@PDA-anti-trop2 NPs were mixed and co-incubated for 30 min, washed with PBS three times, and then imaged confocally to obtain a result shown in FIG. 2, wherein FIG. 2A illustrates a bright-field image, FIG. 2B illustrates a fluorescence image, and FIG. 2C is an image obtained by combining FIG. 2A and FIG. 2B. It can be seen, from FIG. 2, that green fluorescence on the surface of the nano-particle is clear, indicating that the target antibody anti-trop2 was successfully connected to the surface of Fe₃O₄@Au NPs-MBA@PDA NPs.

### Embodiment 3

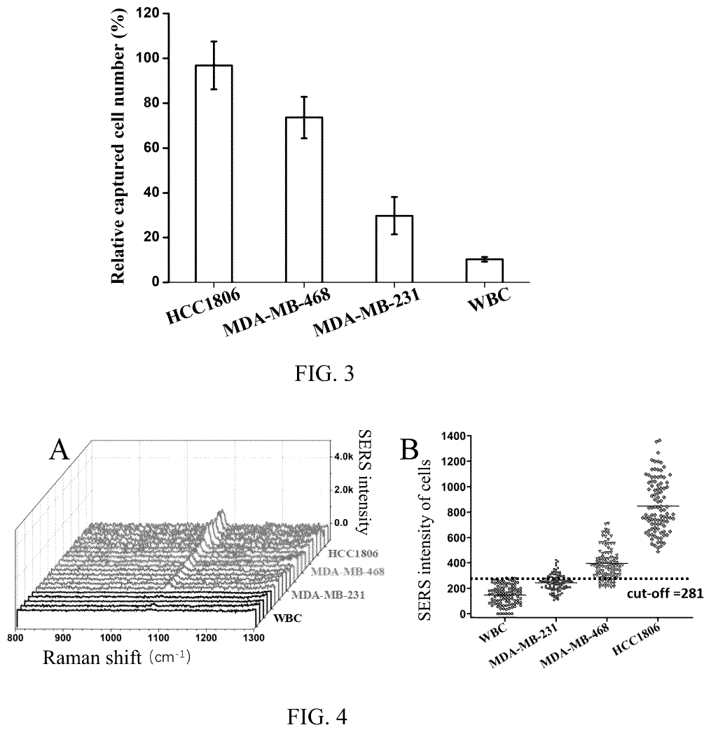

Fe₃O₄@Au NPs-MBA@PDA-anti-trop2 (146 μg/ml) was respectively co-incubated with TNBC cell strains HCC1806 (ATCC CRL-2335), MDA-MB-468 (ATCC HTB-132) MDA-MB-231 (ATCC CRM-HTB-26) with different trop2 expression levels (wherein, HCC1806 had the maximum expression level, and MDA-MB-231 had the minimum expression level) and WBCs with a negative trop2 expression, then cells were captured with a magnet and sufficiently washed with PBS, and then captured cells were counted with a cell counter. As shown in FIG. 3, the TNBC HCC1806 cell capture rate, the TNBC MDA-MB-468 cell capture rate and the TNBC MDA-MB-468 cell capture rate of a magnetic SERS nano-probe are 97%, 74% and 30% respectively, indicating that the capture rate of the nano-probe decreases with the decrease in the trop2 expression level. The capture rate of the nano-probe for WBCs is only 10% and is different from the capture rate for other TNBC cells, indicating that the nano-probe can effectively capture tumor cells with a positive trop2 expression.

### Embodiment 4

Fe₃O₄@Au NPs-MBA@PDA-anti-trop2 (146 μg/ml) was respectively co-incubated with four types of cells HCC1806, MDA-MB-468, MDA-MB-231 and WBCs, and then the four types of cells were secured on a glass slide; 100 cells of each type were selected randomly, and the SERS intensity of each cell was measured with a Raman spectrometer. As shown in FIG. 4A, the average SERS signal intensity of the four types of cells tends to increase with the increase in the trop2 expression level. FIG. 4B shows the SERS intensity of each of the four types of cells. It can be seen from FIG. 4B that the SERS intensity of different cell strains fluctuates within a certain range, leading to an overlap of the SERS intensity ranges of cells of different cell strains; particularly, the SERS intensity of for WBCs overlaps to different extents with the SERS intensity of MDA-MB-231 with a low trop2 expression and the SERS intensity of MDA-MB-468 with a medium trop2 expression, indicating the presence of false positive interference of SERS signals of WBCs, so it is difficult to identify WBCs and tumor cells by directly measuring the SRES signal intensity of a certain cell.

### Embodiment 5

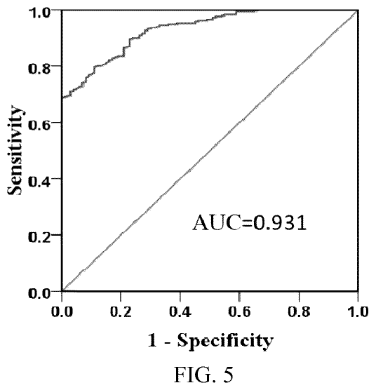

A ROC curve for comparing the SERS intensities of HCC1806, MDA-MB-468, MDA-MB-231 and WBCs was plotted with a SPSS, wherein AUC=0.913 (if AUC=0.5, it indicates the recognition capacity is unavailable; if 0.5<AUC≤0.7, it indicates that the recognition capacity is low; if AUC>0.9, it indicates that the recognition capacity is good), indicating that the SERS intensity has a good capacity to recognize tumor cells, as shown in FIG. 5.

### Embodiment 6

Several thresholds for comparing the SERS intensities of tumor cells and WBCs obtained by analyzing the ROC curve in Embodiment 5 are listed in Table 1. It can be seen, from Table 1, that each threshold corresponds to one sensitivity and specificity. When cut-off=281, the specificity reaches 100%, so the interference from WBCs can be eliminated, and the sensitivity is high.

When one of the sensitivity and specificity increases, the other one of the sensitivity and specificity will decrease. In actual application, the threshold should be selected based on the study purpose and the balance between the sensitivity and specificity. For example, when gene detection of tumor cells is needed later, tumor cells should be accurately distinguished from WBCs. To avoid misdiagnosis to the maximum extent within a permissible range of the rate of missed diagnosis, that is, to guarantee the sensitivity under the precondition of improving the specificity to the maximum extent, the threshold was set to 281, and in this case, the specificity was 100% (the rate of misdiagnosis was 0), and the sensitivity was 69% (the rate of missed diagnosis was 31%). If the detection purpose is primary screening of tumor cells, for example, when CTCs in blood need to be primarily screened for monitoring tumor recurrence of cancer patients, early warning of tumor recurrence can be realized, and in this case, based on the principle of avoiding missed diagnosis within a permissible range of the rate of missed diagnosis, that is, to guarantee the specificity under the precondition of improving the sensitivity to the maximum extent, the threshold was set to 206, the sensitivity was 90% (the rate of missed diagnosis was 10%), and the specificity was 76% (the rate of misdiagnosis was 24).

TABLE 1
Several thresholds for identifying SERS
intensities of tumor cells and WBCs

| Threshold | Sensitivity | Specificity | Purpose |
|---|---|---|---|
| 317 | 60% | 100% | |
| 297 | 65% | 100% | |
| 281 | 69% | 100% | accurate recognition of tumor |
| | | | cells(for example, gene |
| | | | detection of tumor cells) |
| 246 | 80% | 88% | |
| 206 | 90% | 76% | primary screening of tumor |
| | | | cells (for example, early |
| | | | warning of tumor recurrence) |
| 181 | 95% | 64% | |
| 106 | 100% | 34% | |

All aspects, embodiments and features of the invention should be construed as illustrative rather than restrictive from all sides, and the scope of the invention is defined by the claims. Other embodiments, modifications and uses can be obtained by those skilled in the art without departing from the spirit and scope of the invention.

The sequence of the steps of the preparation method provided by the invention is not limited to those listed, and changes to the sequence of these steps made by those ordinarily skilled in the art without creative labor should also fall within the protection scope of the invention. In addition, two or more steps or actions may be performed at the same time.

Finally, it should be noted that the specific embodiments described here are merely used for explaining the invention by way of examples and are not used for limiting the implementations of the invention. Those skilled in the art can make various modifications, supplements or similar substitutions to the specific embodiments described above, and it is unnecessary and impossible to enumerate all implementations of the invention. All these obvious modifications or transformations obtained based on the essential spirit of the invention should also fall within the protection scope of the invention, and it will be against the spirit of the invention to interpret them into any additional limitations.

1. (canceled)

2. (canceled)

3. A SERS nano-particle, wherein the SERS nano-particle sequentially comprises, from inside to outside, core particles, a noble metal nano-particle layer, a Raman signal molecular layer, a high molecular layer and a target antibody anti-trop2 layer;
the core particles are magnetic particles, surfaces of which are provided with a positive-charge polymer modifying layer;
the noble metal nano-particle layer is layered structure formed by noble metal nano-particles assembled on the surfaces of the core particles by means of electrostatic interaction;
the Raman signal molecular layer is a layered structure formed by Raman signal molecules connected to a surface of the noble metal nano-particle layer;
the high molecular layer is a layered structure formed by hydrophilic molecules wrapping a surface of the Raman signal molecular layer;
the target antibody anti-trop2 layer is a layered structure formed by a target antibody anti-trop2 coupled to an outer surface of the high molecular layer.

4. The SERS nano-particle according to claim 3, wherein a particle size of the core particles is 10-1000 nm, a particle size of the noble metal nano-particles is 1-100 nm, and a particle size of the SERS nano-particle is 100-1000 nm.

5. A preparation method for the SERS nano-particle according to claim 3, comprising the following steps:
(S1) obtaining core particles;
(S2) assembling noble metal nano-particles on surfaces of the core particles by means of electrostatic interaction to form a noble metal nano-particle layer to obtain a particle I;
(S3) connecting Raman signal molecules to a surface of the noble metal nano-particle layer to form a Raman signal molecular layer to obtain a particle II;
(S4) wrapping a surface of the Raman signal molecular layer with hydrophilic molecules to form a high molecular layer to obtain a particle III; and
(S5) coupling an antibody anti-trop2 to a surface of the high molecular layer to form a target antibody anti-trop2 layer to obtain a magnetic SERS nano-particle.

6. The preparation method according to claim 5, wherein in (S1), the core particles are obtained by a reaction I of a solution containing a magnetic metal salt, an alkaline substance, a positive-charge polymer and a solvent I.
in (S2), a noble metal nano-particle solution and a core particle solution are mixed, and then stirring I is performed to obtain the particle I;
in (S3), a Raman signal molecule solution and a particle I solution are mixed, and then stirring II is performed to obtain the particle II;
in (S4), a particle II solution and CH₃CH₂OH, NH₃·H₂O are mixed, stirring III is performed, and a polydopamine solution is added for a reaction III to obtain the particle III;
in (S5), the SERS nano-particle is obtained by stirring IV of a solution containing the particle III, a buffer solution and a target antibody anti-trop2.

7. The preparation method according to claim 6, wherein in (S1), the magnetic metal salt comprises a magnetic metal chloride, the alkaline substance comprises CH₃COONa, the positive-charge polymer comprises polyetherimide, the solvent I comprises glycol, and conditions for the reaction I are as follows: a time for the reaction I is 1-10 hrs, and a temperature for the reaction I is 100-500° C.;
in (S2), a time for the stirring I is 10-120 min;
in (S3), a time for the stirring II is 1-10 hrs;
in (S4), a time for the stirring III is 1-100 min, and a time for the reaction III is 1-10 hrs;
in (S5), the buffer solution comprises at least one selected from a Tris-HCl solution and an ammonia alkaline solution, a time for the stirring IV is 1 h-72 hrs, and a temperature for the stirring IV is 15-40° C.

8. A method for distinguishing CTCs from WBCs for a non-diagnostic purpose, comprising: enabling the SERS nano-particle according to claim 3 to make contact with a to-be-detected solution containing CTCs and/or WBCs; performing incubation; then detecting a SERS signal intensity of a cell combined with the SERS nano-particle; setting a SERS signal intensity threshold; if the SERS signal intensity of the cell combined with the SERS nano-particle exceeds the SERS signal intensity threshold, determining the cell as a CTS; otherwise, determining the cell as a WBC.

9. The method for distinguishing CTCs from WBCs for a non-diagnostic purpose according to claim 8, wherein the SERS signal intensity threshold is a point value within a fluorescence intensity of 0-3000a.u.

10. The method for distinguishing CTCs from WBCs for a non-diagnostic purpose according to claim 8, wherein the SERS signal intensity threshold is determined according to a ROC curve of SERS intensities of CTCs and WBCs in known samples.

11. The method for distinguishing CTCs from WBCs for a non-diagnostic purpose according to claim 8, wherein SERS intensities of CTCs and WBCs in known samples are input to a SPSS, and the SERS signal intensity threshold is obtained after analysis of a ROC curve.

12. A system for distinguishing CTCs from WBCs for a non-diagnostic purpose, comprising:
a sample loading module, used for collecting and/or storing samples, wherein the samples comprise known samples formed by certain CTCs and WBCs and/or to-be-detected samples formed by uncertain CTCs and WBCs;
a ROC curve construction module, used for detecting SERS signal intensities of the known samples, plotting an ROC curve of specificity and sensitivity, and outputting a recommended threshold according to detection requirements; and
a detection module, used for detecting SERS signal intensities of the to-be-detected samples and outputting a detection result identifier according to the set threshold.

13. The SERS nano-particle according to claim 3, wherein a positive-charge polymer in the positive-charge polymer modifying layer comprises polyetherimide.

14. The SERS nano-particle according to claim 3, wherein the magnetic particles comprise at least one selected from Fe nano-particles, FeO nano-particles and Fe₃O₄ nano-particles;
the noble metal nano-particles comprise at least one selected from gold nano-particles, silver nano-particles, platinum nano-particles and nano-copper particles;
the Raman signal molecules comprise at least one selected from 4-mercaptobenzoic acid, mercaptopyridine, 4-aminothiophenol, naphthalenethiol, 4-fluorothiophenol, rhodamine, crystal violet, alizarin red and Nile blue;
the hydrophilic molecules comprise at least one selected from polydopamine, bovine serum albumin and polyethylene glycol.

15. The method for distinguishing CTCs from WBCs for a non-diagnostic purpose according to claim 11, wherein SERS intensities of CTCs and WBCs in known samples are input to a SPSS to plot a ROC curve, a series of cut-off values corresponding to different sensitivities and specificities are obtained after the ROC curve is analyzed, and a cut-off value corresponding to a maximum specificity and sensitivity is the SERS signal intensity threshold.
